(19) United States
(12) Patent Application Publication
(10) Pub. No.: US 2005/0143629 A1
(43) Pub. Date: Jun. 30, 2005

(54) Method for a classification guilty knowledge test and integrated system for detection of deception and information

(76) Inventor: Lawrence A. Farwell, Seattle, WA (US)

Correspondence Address: Brainwave Science Inc.;Attn: Bruce Lisanti, Suite 402, 4616 25th Ave. NE, Seattle, WA, 98105, US

(21) Appl. No.: 10/872,967

(22) Filed: Jun. 21, 2004

Related U.S. Application Data
(60) Provisional application No. 60/479,932, filed on Jun. 20, 2003.

(51) Int. Cl. A61B 5/00; A61B 5/04; A61B 10/00; G06F 17/00

(52) U.S. Cl. 600/300; 600/544; 128/920

(57) ABSTRACT

A method for detecting deception or information possessed by a subject is disclosed. A subject is presented with known relevant stimuli, irrelevant stimuli, and critical relevant stimuli. Psychophysiological responses to the stimuli are measured and classified. A conclusion regarding the presence or absence of information is drawn from the classification. Many alternatives are disclosed such as measuring central nervous system responses to enhance the conclusion.

### CROSS-REFERENCE TO RELATED APPLICATIONS

This application claims the benefit of U.S. Provisional Application No. 60/479,932, filed Jun. 20, 2003, by the present inventor.

### BACKGROUND

Conventional polygraphy typically uses three basic kinds of tests: a relevant/irrelevant (R/I) test, a control question test (CQT), and a guilty knowledge test (GKT).

In relevant/irrelevant tests, a subject under investigation is classified after being asked two types of questions: relevant questions and irrelevant questions. A known relevant question is relevant to the crime or situation under investigation (e.g., “Did you shoot Jones last Thursday?”). The subject under investigation may know correct answers to a relevant question. The subject matter of known relevant questions may be known to the subject through an event, such as an interrogation or reading the newspaper, that preceded the test. Alternately, known relevant questions may address details about a crime that are intentionally revealed to the subject immediately before the test. Relevant questions provide a standard response for questions known to be relevant to the situation or known by the subject. Conversely, an irrelevant question is irrelevant to the situation under investigation. Irrelevant questions may contain incorrect details about the crime, but the incorrect details may appear to be plausible for a subject who lacked detailed knowledge about the crime (e.g., an innocent subject who was not at the scene of the crime). Irrelevant questions provide a standard response for questions known to be irrelevant to the crime or situation under investigation.

In a relevant/irrelevant test, responses to relevant questions are compared to responses to irrelevant questions. If the responses to the relevant questions are significantly larger than the responses to the irrelevant questions, the subject is determined to be lying. If the responses to the relevant questions are smaller than, equal to, or slightly larger than the responses to the irrelevant questions, the subject is determined to be truthful. Such a test utilizing relevant and irrelevant questions can negatively impact an innocent subject's response by allowing a relevant question to disrupt an innocent subject's response. In one such embodiment, an innocent subject's response to a relevant question may produce an answer that is relevant for the wrong reasons (e.g., relevant for reasons other than being relevant to the situation or known by the subject). In addition, there may be other variables unrelated to guilt or lying, such as the emotional qualities of the subject, which affect the response. If there is a large response to the relevant questions, it may simply result from an emotional subject who is telling the truth. If there is no marked response to either the relevant or irrelevant questions, it may be possible that the subject is lying but that he or she is physiologically unresponsive. Neither a large response nor a lack of a response provides a clear-cut, unique interpretation that sheds light on the truthfulness of the subject. For this reason, the R/I test, although common in the early days of polygraphy, is rarely used today.

The control question test (CQT) was developed in an attempt to overcome the lack of control in the R/I test. In the CQT, the subject is asked control questions in addition to relevant and irrelevant questions. A control question is designed to elicit a stress response in the subject regardless of whether the subject is innocent/truthful or guilty/lying (e.g., “Before the age of 21, did you ever lie to someone who trusted you?”) If the subject is deceptive, he or she may be more emotionally aroused (and consequently more physically aroused) by the relevant questions, whereas if the subject is truthful, he or she may be more emotionally and physically aroused by the control questions. (The irrelevant questions are irrelevant to the investigated situation, and are not included in the analysis.) A larger response to the relevant questions yields a “deceptive” determination. A larger response to the control questions yields a “nondeceptive” determination.

Although the CQT presents three types of stimuli, it uses only two types of stimuli in the analysis: the control and relevant questions. Responses to the irrelevant questions are ignored. The CQT compares responses to relevant questions to the responses to control questions. A CQT may be advantageous over an R/I test since the CQT presents a type of question, other than the relevant stimuli, that may elicit a marked response in the subject. The R/I test provides only a comparison of a stress response with a lack of a stress response. The CQT allows for the comparison of a stress response with another stress response, with the latter (control question) response designed to be elicited regardless of whether the subject is deceptive or truthful. For this reason, the CQT has significant advantages over the R/I test, and is consequently much more widely used today.

One difficulty with the CQT is that the control questions are fundamentally different from the relevant questions. This makes comparison between the responses problematic. If an examiner develops control questions that are mild and inoffensive, then the subject will tend to have small responses to the control questions. Since relevant questions, as a result of their content, may be inherently upsetting for a subject even if he or she is truthful, the responses of a truthful subject to the relevant questions may be larger than the responses to the control questions, resulting in a false determination of “deceptive” for a truthful subject. If, on the other hand, the examiner develops control questions that are highly arousing to the subject, then the subject may tend to have a large response to the control questions. If this response is larger than the response to the relevant questions, the subject may be determined to be nondeceptive. Thus, a test in which the control questions are highly provocative may tend to make subjects look nondeceptive even if they are deceptive.

Another difficulty of the CQT lies in the nature of the control questions. In order for the control questions to be effective in eliciting a stress response, the examiner must deceive the subject about the control questions. Subjects are generally told that they will hear questions about the investigated situation (the relevant questions) along with other questions that, if they respond strongly to them, will indicate that they are “the kind of person who would commit this kind of crime.” These other questions are the control questions, although they are not called that in the description to the subject. (In the instructions to the subject, the polygraph examiner will often refer to the irrelevant questions as “control” questions, although they do not serve this function and are not included in the analysis.) In effect, the subjects are told that the control questions are another kind of relevant question. In other words, subjects are led to believe that a large response to control questions will make them look guilty/deceptive, whereas the truth is that a large response to the control questions will make them look innocent/truthful.

It is necessary for this deception of the subject by the examiner to work in order for the control questions to have their desired function. If a subject does not believe the examiner and sees the control questions for what they are—questions that are not of genuine concern but asked to produce a stress response for comparison purposes, the test does not work properly. In general, one of two things will tend to happen. First, the subject, knowing the control questions are not of genuine concern in the investigation, may be unconcerned with the control questions and will produce a small response to them. Since the relevant questions are inherently distressing due to their subject matter, the subject may tend to produce a larger response to the relevant questions than to the control questions, resulting in a “deceptive” determination even if the subject is truthful. Also, the subject, knowing that responses to the control questions will be compared to the relevant responses, may attempt to produce a large response to the control questions, and if successful, will appear nondeceptive regardless of whether the subject is deceptive or not.

In addition, a subject who knows how the test works and who can recognize the control and relevant questions, can enhance their response to the control questions through countermeasures (e.g., biting the tongue or thinking of something highly emotionally distressing). If the subject succeeds in producing a larger response to the control questions than their response to the relevant questions, the subject may be found nondeceptive even if they are deceptive, and even if they do not in any way suppress their response to the relevant questions. All of these difficulties with the CQT arise from the fact that the control questions are fundamentally different from the relevant questions. Thus, control questions do not provide an adequate basis for comparison, or at least not an ideal basis for comparison.

The GKT, like the R/I test, has two types of stimuli: relevant and irrelevant questions. The difference between the GKT and the R/I test is that in the GKT, the relevant questions are relevant to specific details about the investigated situation, known only to the perpetrator and examiners. Thus, in the GKT an innocent and truthful subject does not know which questions are relevant. This eliminates one of the two major problems with the R/I test, namely that truthful subjects will show large emotional and physiological responses to the relevant questions due to the distressing content of the questions and not due to the subject being deceptive.

In the GKT, the subject is informed of certain categories of information regarding the investigated situation. For example, the subject is told that one item that he or she will be questioned about will be the murder weapon. The subject is also told that they will be asked about several alternatives as to what the correct item in this category is (e.g., “knife,” “pistol,” “rifle,” “ice pick,” “baseball bat,” “rope”). The subject is not, however, told what the actual murder weapon is.

The questioning in the GKT may be of the following form. (Here, assume that the authorities know that the murder weapon was a rifle, and the suspect claims not to know what the murder weapon was.)

“Regarding the murder weapon, do you know that it was a:”
“knife” (irrelevant)
“pistol” (irrelevant)
“rifle” (relevant)
“ice pick” (irrelevant)
“baseball bat” (irrelevant)
“rope” (irrelevant)

All subjects are expected to truthfully answer “no” to the irrelevant questions. An innocent subject is expected to truthfully answer “no” in response to the relevant question. A subject who knows the details about the crime is also expected to answer “no” to the relevant question, but in his case this will be a lie.

The premise of the GKT is that the subject who knows the details about a crime will emit a larger stress response when he lies in response to questions about the correct alternatives (e.g., “rifle”) than when he tells the truth in response to questions about the incorrect alternatives (e.g., “pistol”) regarding relevant details of the crime. A subject who is innocent of the crime and does not know the details of the crime will not know which items are correct details. He will not know which are the relevant questions. Therefore, he is not expected to emit a larger response to the relevant items.

Data analysis in the GKT consists of comparing the responses to the relevant and irrelevant questions, and determining if the relevant responses are larger than the irrelevant responses. Since an innocent and truthful subject will not know which are the relevant questions, it is unlikely that a truthful subject will emit a large stress response to the relevant questions and be falsely determined to be deceptive. This addresses one of the two major problems with the original R/I test.

The primary remaining problem with the GKT is the same as the second major problem with the R/I test: what if a subject does not respond to any of the stimuli? Does this mean he does not know the details about the crime or is it just that he is physically and/or emotionally unresponsive, and would not respond even if confronted directly with a bloody murder scene where he had been the perpetrator? What would it have taken to make him respond? The GKT, like the R/I test, provides no answers to these questions, and no relevant data with which to address them. A negative result in the GKT, like a negative result in the R/I test, is uninterpretable from a scientific standpoint.

A second major problem with the GKT is the same as one of the major problems with the CQT, and that is susceptibility to countermeasures. Just as a person can fake a stress response to the control questions in the GKT, he can fake a stress response to one of the irrelevant questions in each series in the GKT. All a deceptive subject has to do to appear truthful in the GKT is to make sure that in each series of questions, his response to one of the irrelevant questions is greater than his response to the relevant question. This can be accomplished through physical countermeasures such as biting the tongue or through emotional countermeasures such as thinking of something highly distressing.

One difficulty with the GKT is that there is no control condition. Like the R/I test, the GKT compares the response of interest (e.g., the response to the relevant questions) only to data that lack a response (e.g., the responses to the irrelevant questions). There is no comparison of the response of interest to a known response of the kind that would be expected if the subject is deceptive.

Recall that the CQT was developed to address this shortcoming of the R/I test. One could add to the GKT a control question of the type used in the CQT, but this would result in the same difficulties it presents in the CQT. (See the above discussion.) The CQT control questions are fundamentally different from the relevant questions, and therefore, do not provide an adequate basis for comparison. This would be the case with the same kind of control questions if they were to be added to the GKT.

### BRIEF DESCRIPTION OF THE DRAWINGS

FIG. 1 is a block diagram illustrating an apparatus in accordance with one embodiment of the invention.

FIG. 2 is a block diagram illustrating a system employing the apparatus of FIG. 1.

FIG. 3 is a data structure diagram illustrating an example of test data for a subject.

FIG. 4 is a flow diagram illustrating an example of an inquiry employing stimuli under one embodiment of the invention.

### DETAILED DESCRIPTION

The invention will now be described with respect to various embodiments. The following description provides specific details for a thorough understanding of, and enabling description for, these embodiments of the invention. However, one skilled in the art will understand that the invention may be practiced without these details. In other instances, well-known structures and functions have not been shown or described in detail to avoid unnecessarily obscuring the description of the embodiments of the invention.

It is intended that the terminology used in the description presented below be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this Detailed Description section.

A method for detecting deception or information possessed by a subject is disclosed. The subject is presented with three types of stimuli: a known relevant stimulus that is relevant to the situation under investigation, an irrelevant stimulus that is irrelevant to the situation under investigation, and a critical relevant stimulus that is relevant to the situation under investigation and that the subject does not acknowledge knowing. Under one embodiment, an autonomic-nervous-system-based (ANS) psychophysiological response and a central-nervous-system-based (CNS) psychophysiological response are measured. An algorithm combines both ANS and CNS data to make a single determination regarding at least one of a) deception, b) guilty knowledge, and c) information possessed by the subject. In addition, the subject is presented with stimuli in at least one of a) the auditory modality and b) the visual modality, wherein the stimuli are generated by a computer that presents the stimuli with a precisely determined time course. A psychophysiological response to the stimuli is measured and classified. The presence or absence of information is determined from the classification.

Much of the following detailed description provided is explicitly disclosed in the provisional patent application noted above; much of the additional material of aspects of the invention will be recognized by those skilled in the relevant art as being inherent in the detailed description provided in such provisional patent application, or well known to those skilled in the relevant art. Those skilled in the relevant art can implement aspects of the invention based on the detailed description provided in the provisional patent application.

Limitations of Prior Designs

A fundamental difficulty with the CQT, the GKT, and the R/I test is that all three depend on a comparison between two types of responses. The responses to two types of questions are compared, and a conclusion is drawn based on a judgment regarding which is “larger” or “more of a response”. This is problematic because there is no clear, universal definition of what “larger” means, or how much larger a response must be to be considered “larger,” or in some cases, even which direction of a response makes it “more of a response.”

The decision that the examiner must make for the CQT is: “Are the responses to the relevant questions larger than the responses to the control questions?” For the GKT and the R/I, the question is identical but the type of response to which the “relevant” responses are compared is different. The question for the GKT and the R/I test is: “Are the responses to the relevant questions larger than the responses to the irrelevant questions?” In all three cases, CQT, GKT, and R/I test, a two-way comparison is made regarding which response is larger or more pronounced. In none of these cases is there a precise, universal, fully articulated methodology for determining whether one response is a “larger” than another, or, if it is larger, how much larger it needs to be before it is considered to be meaningfully different.

Note that in the CQT, three types of questions are asked, but only two are analyzed—the relevant and control responses are compared, and the irrelevant responses do not contribute to the analysis. The problem could not be solved by introducing a classification because the control questions are not the same type of questions as the relevant questions. Although they are designed to be emotionally distressing and consequently physically arousing, they refer to a different type of subject matter than the relevant questions. The relevant questions are also a fundamentally different type of question from the irrelevant questions, whether the person is truthful or deceptive. The relevant questions are about inherently emotionally distressing subjects (e.g., the subject of the interrogation), whereas the irrelevant questions are about emotionally neutral matters. Thus it would not be valid to attempt to classify the responses to the relevant questions with respect to the control questions and/or the irrelevant questions.

In order to employ the statistically powerful and scientifically sound methodology of classification, one must have at least two standard categories, and a third type of data that are to be classified as belonging to one category or another. Obviously, classification techniques cannot be used in the conventional GKT because there are only two types of stimuli.

In the CQT data analysis, to reach a determination of deceptive or nondeceptive, relevant and control questions are compared to see which has the larger response, and irrelevant questions are not included in the analysis. Two types of responses, the response to the relevant questions and the response to the control questions, are compared. No attempt is made at classification, nor would such an attempt be valid if it were tried, for reasons described above and further elaborated below.

In the CQT, the three types of questions have the following characteristics. Control questions are designed to be a) emotionally arousing and b) irrelevant to the crime. Irrelevant questions are designed to be a) emotionally neutral and b) irrelevant to the crime. Relevant questions are designed to be a) emotionally arousing and b) relevant to the crime for a deceptive subject, and a) not highly emotionally arousing and b) relevant to the crime for a nondeceptive subject. It would not be valid to attempt to implement a classification methodology with such a data set. The characteristics of the relevant questions do not match the characteristics of either the control questions or the irrelevant questions, whether the subject is deceptive/guilty or truthful/innocent.

If the subject is deceptive, the relevant questions are similar to the control questions in that they are designed to be emotionally arousing, but are different in that they are not relevant to the crime. If the subject is nondeceptive, the relevant questions are designed to be similar to the irrelevant questions in that they are expected not to be highly emotionally arousing, but they are different from the irrelevant questions in that they are relevant to the crime, and this will no doubt make the relevant questions highly emotionally arousing for some subjects even if they are innocent and nondeceptive. Thus, classification techniques, and the statistical power and scientific rigor they bring, are not applicable in the CQT.

For these same reasons, even a comparison between relevant and control questions, without an attempt at classification, can be problematic from a scientific perspective. The relevant and control questions are of fundamentally different types, are not directly comparable, and may elicit fundamentally different responses regardless of deception or truthfulness.

In the currently practiced polygraph techniques, the response of the subject depends not only on the questions, but on a number of other factors that are outside the interrogator's knowledge or control. One such factor is how much the subject knows about how the test works (e.g., whether or not he believes that the control questions are of genuine concern, or realizes that they are control questions inserted for the sake of comparison with the relevant questions). Thus, even if the questions were perfectly designed, responses could not be unambiguously interpreted. This is described in more detail below.

All of the current polygraph techniques, R/I tests, CQT, and GKT, involve comparing responses to two types of stimuli, determining which response is “larger,” and drawing conclusions regarding the credibility of the subject based on this comparison.

Two fundamental problems are inherent in this process. First, there is no clear, unambiguous definition of what is a “larger” physiological response. For example, what if the skin conductance response is larger (however defined) to the relevant questions, and the cardiovascular response is larger (however defined) to the control (CQT) or irrelevant (GKT or I/R) questions? What if the skin conductance response is larger to the relevant questions, but only 0.00001% larger? Second, even if the physiological differences are clear, there is no unambiguous interpretation of the physiological responses that provides unambiguous evidence regarding the subject's veracity.

a. For the CQT, if the responses to the relevant questions are larger than the responses to the control questions, this may mean either:
i. The subject is deceptive/guilty, and is therefore more aroused by the control than relevant questions (true positive).
ii. The subject is truthful/innocent, but the control questions are too mild, so the subject responds more to the relevant questions due to their inherently disturbing nature (false positive).
iii. The subject is truthful/innocent, the control questions are appropriate, but the subject knows how a CQT test works, recognizes the control questions as not being of genuine concern and hence emits small responses to the control questions, and emits large responses to the relevant questions due to their inherently disturbing nature (false positive).

b. For the CQT, if the responses to the control questions are larger than the responses to the relevant questions, this may mean either:
i. The subject is truthful/innocent, the control questions are appropriately distressing, the subject is not unduly aroused by the relevant questions, and therefore the subject is more aroused by the control than relevant questions (true negative).
ii. The subject is deceptive/guilty, but practices countermeasures to enhance his response to the control questions such that they are larger than the responses to the relevant questions (false negative).
iii. The subject is deceptive/guilty, but the control questions are too strong, so the subject responds more to the control questions than the relevant questions (false negative).
iv. The subject is deceptive/guilty, but is for some reason (e.g., cultural or psychological differences) is emotionally unconcerned with the relevant questions, and consequently does not emit a large response to them (false negative).

c. For the GKT or R/I test, if the responses to the relevant questions are larger than the responses to the irrelevant questions, this may mean either:
i. The subject is deceptive/guilty, and therefore the subject is more aroused by the relevant than irrelevant questions (true positive);
ii. The subject is truthful/innocent, but (for the R/I test) he is more aroused by the relevant than irrelevant questions due to the inherently distressing nature of the relevant questions (false positive). False positives are unlikely in the GKT, because the truthful/innocent subject does not know which questions are the relevant questions.

d. For the GKT or R/I test, if the responses to the relevant questions are not larger than the responses to the irrelevant questions, this may mean either:
i. The subject is truthful/innocent, and therefore is not emotionally aroused by the relevant questions (true positive);
ii. The subject is deceptive/guilty, but practices countermeasures to enhance his response to one or more of the irrelevant questions such that they are larger than the responses to the relevant questions (false negative);
iii. The subject is deceptive/guilty, but for some reason is emotionally unresponsive to the relevant questions (false negative);
iv. The subject is deceptive/guilty, but for some reason is physically unresponsive to the relevant questions (or, perhaps, to any possible question) (false negative).

The Role of Brain Fingerprinting® Science

The application of Brain Fingerprinting® (one embodiment described herein) or other techniques in a criminal case involves three phases: 1) investigation, including evidence gathering and interviews; 2) scientific testing; and 3) adjudication. Of these three phases, only the second phase is in the domain of science. The first phase is undertaken by skilled investigators, including skilled interviewers who may be scientists as well, the second by a scientist, and the third by a judge and jury.

This is similar to the forensic application of other sciences. For example, if a person is found dead of unknown causes, first there is an investigation to determine if there may have been foul play. If there is a suspect involved, the suspect is interviewed to determine what role, if any, he says he has had in the situation. If the investigation determines that the victim may have been poisoned using ricin or cadmium, two rare and powerful poisons, then scientific tests can be conducted to detect these specific substances in the body. Then the evidence accumulated through the test, the investigation, and the interview are presented to a judge and jury, who make the adjudication as to whether a particular suspect is guilty of a particular crime.

In such a case, the science of forensic toxicology reveals only whether or not specific toxins are in the body. It does not tell us when or where to look for toxins, or which toxins to look for. We must rely on investigation to provide the necessary guidance on these issues. The science of forensic toxicology also does not tell us whether a particular suspect is innocent or guilty of a crime. The question of guilt or innocence is a legal one, not a scientific one, and the adjudication is made by a judge and jury, and not by a scientist or a computer.

Phase 1: Investigation

The first phase in applying Brain Fingerprinting® (or other techniques described herein) in a criminal case is an investigation of the crime. Before a Brain Fingerprinting® test can be applied, an investigation must be undertaken to discover information that can be used in the test and to identify an appropriate suspect or suspects. The science of Brain Fingerprinting® accurately determines whether or not specific information is stored in a specific person's brain. It detects the presence or absence of specific information in the brain. Before we can conduct this scientific test, we need to determine what information to test for and whom to test. This investigation precedes and informs the scientific phase which constitutes the Brain Fingerprinting® test itself.

The role of investigation is threefold: 1) to discover salient information about the crime, which will constitute the information to be used in a Brain Fingerprinting® test; 2) to identify one or more suspects; and 3) to determine what crime-relevant information the suspect has been exposed to outside of committing the crime (e.g., through news media, interrogation, or a previous trial), and what crime-relevant information the suspect denies knowing or recognizing as significant.

As with any scientific test, if the outcome of the Brain Fingerprinting® test is to be useful evidence for a judge and jury to consider in reaching their verdict, then the information tested must have a bearing on the perpetration of the crime. The job of the investigator is to find features relevant to the crime that have the following attributes.

1. They are salient features that perpetrator encountered in the course of committing the crime, such as the murder weapon or the specific location of the crime.

2. The suspect denies knowing the information they contain in the context of the crime (or other event, action, or knowledge at issue).

3. The suspect denies recognizing them as significant or as different from items of the same type that are irrelevant to this crime.

4. Either the suspect has not been exposed to them in some other context, such as interrogation, court proceedings, or news media reports, or any such exposure has been minor enough that the suspect claims not to know the information embodied in the probes.

These features of the crime will be used in the Brain Fingerprinting® test as probe stimuli. If the suspect knows these specific features of the crime, and has had no access to this information other than through committing the crime, then this will provide evidence of his involvement in the crime. If the suspect lacks this knowledge, this will provide evidence supporting his innocence. Brain Fingerprinting® tests for the presence or absence of this information stored in the suspect's brain.

The investigation often includes interviews of witnesses to gather evidence. It may include interviews of multiple suspects. Before the scientific phase, the Brain Fingerprinting® test, can be undertaken, at least one suspect must be identified, and this suspect must be interviewed to determine if it is possible to structure the probe stimuli that meet the criteria necessary for a Brain Fingerprinting® test.

Moreover, once evidence has been accumulated, and before the scientific test is conducted to determine if the evidence can be linked to the suspect, it can in some cases be very valuable to obtain the suspect's account of the situation. For example, if an investigation shows that specific fingerprints are found at the scene of a murder, a suspect can be interviewed to determine if there may be some legitimate reason that his prints are there. If the suspect's story is that he was never at the scene of the crime, then a match between his fingerprints and the fingerprints at that scene would be highly incriminating. If, on the other hand, the suspect's story is that he was at the scene for some legitimate reason just before the crime, then fingerprints must be interpreted differently, particularly if there is corroborating evidence of the suspect's presence at the scene before the crime.

The interview with the suspect may help to determine which scientific tests to conduct, or how to conduct the tests. For example, a suspect may say that he entered and then left the room where a murder was committed a short time before the murder, and that he never saw or handled the murder weapon. In this context, a finding that the suspect's fingerprints matched the fingerprints on the doorknob would have little value, but a finding that his fingerprints matched those on the murder weapon would provide incriminating evidence.

Prior to a Brain Fingerprinting® test, an interview of the suspect is conducted. The suspect is asked if he would have any legitimate reason for knowing any of the information that is contained in the potential probe stimuli. This information is described without revealing which stimuli are probes and which are irrelevants. For example, the suspect may be asked, “The newspaper reports, which you no doubt have read, say that the victim was struck with a blunt object. Do you have any way of knowing whether that murder weapon was a baseball bat, a broom handle, or a blackjack?” If the suspect answers “No,” then a test result indicating that his brain does indeed contain a record of which of these is the murder weapon can provide evidence relevant to the case. In this case, “baseball bat” (the actual murder weapon) could become a probe stimulus.

The suspect may have been exposed to information about the crime through an error on the part of interrogators in previous interrogations, through familiarity with the scene or the victim that has nothing to do with the crime, through hearsay from people directly or indirectly involved, or through any number of other channels. The suspect is to be given a chance before the Brain Fingerprinting® test to disclose any familiarity he may have with the crime, so that any probes that he knows about for a legitimate reason can be eliminated from the test. Probes or critically relevant stimuli contain crime-relevant information that the suspect claims to have no way of knowing except through having been present at the crime, and that the suspect or subject denies knowing.

The targets are also discussed in the interview. Targets or relevant stimuli contain information about the crime that the suspect knows whether he committed the crime or not, and are used to establish a baseline brain response for information known to be significant to this subject in the context of the crime. For example, if media reports known to the suspect have disclosed the location of a murder (e.g., “behind Newton Stadium”), then the location of the murder could be used as a target stimulus. In the interview, the interviewer makes sure that the suspect does indeed know the information contained in the target stimuli.

In the interview, the suspect is also given a list of all of the stimuli to be presented in the test, without disclosing which stimuli are probes and which are irrelevant. The suspect is asked to identify any stimuli that are significant to him for reasons that have nothing to do with the crime. If any stimulus is significant to the suspect for reasons having nothing to do with the crime, then that stimulus is eliminated from the test. For example, if the suspect claims to know nothing about who committed the crime and to be unfamiliar with any of the other people who are suspected of being involved, one of the probe stimuli may be the name of a known accomplice. If that name also happens to be the name of the suspect's brother-in-law, then the stimulus would be significant to the suspect for reasons unrelated to the crime, and this stimulus would be eliminated from the test. In this way, the interview sets the stage for later interpretation of the test. If the suspect has just told investigators that none of the stimuli are significant to him for any reason, and then the probe stimuli are revealed through Brain Fingerprinting® to be significant to the suspect in the context of the crime, this scientific finding provides evidence relevant to the suspect's involvement in the crime.

A logical question is, “What if one of the probe stimuli is significant to the suspect for the wrong reason?” The interview serves to eliminate this potential confound, as described above. Things are significant to a person in context. The context of the probe stimuli in relation to the crime is established in the interview, and prior to the Brain Fingerprinting® test. Immediately before each test, the context of the probe stimuli is established, and the probe stimuli are described without being specifically named. For example, the subject is told, “In this test, you will see several items, one of which is the murder weapon.” Then several different stimuli are presented, including the murder weapon (e.g., “baseball bat”) and several other options that would be equally plausible for an innocent person not familiar with the crime (e.g., “broom handle”). We all have some familiarity with baseball bats and broom handles, but in the context of the murder in which the baseball bat was the murder weapon “baseball bat” will be significant for the perpetrator and not for an innocent suspect who does not know what the murder weapon was. Thus, categories of questions or stimuli are created, and the category is presented to the suspect before the stimuli.

In some cases, the interview will reveal that the suspect is already familiar with all of the information that might be used to structure probe stimuli, for some reason unrelated to committing the crime. For example, interrogators may have mistakenly revealed to the suspect all that they know about the crime. In such a case, there is no remaining information to be used to structure probe stimuli, and a Brain Fingerprinting® test is not conducted. If a Brain Fingerprinting® test were conducted to test whether or not a suspect knew details of a crime that had been told to him by investigators, the test would result in an “information present” result, which would be correct. The probative value of such a result with respect to committing the crime, however, would be nil. Such a test would only reveal, correctly, that the suspect knows the information. Since the investigators already know for certain that the suspect knows the information—because they told it to him—this result would not provide any useful evidence with respect to solving the crime. This is why a test is not conducted under such circumstances. Note that this is a limitation only on when Brain Fingerprinting® can be usefully applied, and not a limitation on the scientific accuracy or validity of the technique. As with DNA, fingerprints, and every other science, there are situations where a correct Brain Fingerprinting® result is simply not useful in solving a particular crime.

In short, Brain Fingerprinting® determines scientifically and accurately what information is stored in a person's brain. It does not determine how that information got there. In order for Brain Fingerprinting® to be useful in identifying a perpetrator—that is, in order for a correct “information present” Brain Fingerprinting® result to be useful evidence regarding a suspect's participation in a crime—investigators must first discover information that would be known to a perpetrator but not to an innocent suspect, and ensure that the subject in question claims not to have obtained that information through some means other than participation in the crime. The interview contributes to this process.

Science is useful only when applied appropriately and intelligently. If the aim is solving a crime or informing a judicial decision, then it is not useful to conduct scientific testing in a vacuum, absent any consideration of what the results will mean in the context of the crime. A test or inquiry is to be structured that will provide relevant and useful results. Also, once the results have been obtained, they are useful only if they are interpreted in the light of the other available evidence, as in the above example regarding fingerprints. Brain Fingerprinting® is like all other sciences in this regard.

The interview serves to refine the selection of stimuli so that the test results will provide useful and relevant information, to establish the relevance of the stimuli, to eliminate potential confounds in the scientific test, and to provide a background for interpretation of the test results once they are obtained.

Conducting the scientific phase of Brain Fingerprinting® depends on a successful outcome of the investigation. To proceed to the scientific phase of the process, the Brain Fingerprinting® test itself, first there must be a successful outcome of the investigation. Without probe or critical relevant stimuli that meet the necessary criteria, a Brain Fingerprinting® test is not possible, just as no fingerprint or DNA tests can be undertaken without fingerprints or DNA. In some cases, the investigation will reveal no information about the crime that would be known only to the perpetrator and to investigators. For example, in the case of a disappearance, investigators may not even know if a crime has been committed, and if so, what were the specific details of the crime. In some sexual assault cases, there may be agreement between the alleged victim and the suspect as to all of the events that took place, but disagreement as to the intent of the parties. Investigators may have made the mistake of revealing to the suspect all that they know about a case. In such cases, no probe stimuli can be developed that will provide evidence relevant to the discrimination between a person who participated in the crime and one who did not. Without such probe stimuli, no Brain Fingerprinting® test can be conducted.

Sometimes it is not possible to find probe stimuli that the investigator can be absolutely certain the suspect has never been exposed to. In some cases it is possible to conduct a meaningful and useful Brain Fingerprinting® test even if the suspect may have had some exposure to the probe stimuli after the crime, e.g., during a trial. The investigator selects the probes to be highly salient features of the crime that the perpetrator encountered in the course of committing a crime, such that a judge and jury are likely to conclude that a perpetrator would know these details. It may be that some details of the crime that would be very salient to an individual committing the crime might not be noticed or taken note of by an innocent suspect sitting through a lengthy trial where such details may have been contained in court documents, photos, testimony, etc. In the context of a trial, an innocent suspect's attention would be on the court proceedings, on convincing the jury of his innocence, etc., and not necessarily on the myriad specific details of the crime, even details that would be very salient for the person committing the crime. The innocent suspect might not even see documents or photos that were passed from a prosecutor to a defense attorney and included in the court records, and might not hear or pay attention to all the details of testimony. Even something that appeared in the news media may not have been seen or heard by the suspect.

In a case where there may be some reference to the information contained in the probe stimuli in court documents or other material that the suspect may have seen or heard, the interview takes on additional importance. It is necessary for the investigator to make certain that the suspect clearly claims not to know the information contained in the probes for any reason unrelated to committing the crime. If the Brain Fingerprinting® test shows that the suspect indeed does not know the crime-relevant information, i.e., if the determination is “information absent,” and if the judge and jury believe that the perpetrator of the crime would know this information, then the “information absent” Brain Fingerprinting® result provides exculpatory evidence. An “information absent” result shows that the suspect indeed does not know the crime-relevant information for any reason, and if the probes are sufficiently salient to convince the jury that the perpetrator would know them, then this result lends itself to unambiguous interpretation by the jury, i.e., that the suspect is not the perpetrator.

An “information present” determination under these circumstances, however, must be interpreted with caution. Particularly when there is some possibility of exposure, it is very important that the investigator obtain a certain and unambiguous statement before the test from the suspect that he does not know the information contained in the probes for any reason unrelated to committing the crime. If the suspect gives any indication that he may know the information contained in the probes for reasons unrelated to committing the crime, then an “information present” determination would not be unambiguously interpretable. That is, the suspect could later claim that he knew the crime-relevant information from exposure after the crime. For this reason, unless the suspect clearly states that he does not know the probes for any reason unrelated to committing the crime, the probes in question must be eliminated. If no unambiguously interpretable probes—i.e., probes that the suspect unequivocally denies knowing—are available, then a Brain Fingerprinting® test cannot be conducted.

The investigator uses his skill and judgment in discovering and evaluating information to be used in the Brain Fingerprinting® test, and any other evidence he uncovers. There is always a degree of uncertainty in this process. If the investigator finds a gun lying by the body of a person who apparently died of gunshot wounds, he may conclude that it is the murder weapon. There is always a possibility, however, that it is not. For example, perhaps the perpetrator shot the victim with one gun and planted another gun to frame someone else. Ultimately, the evidence accumulated by the investigator will need to be weighed by the judge and jury for its bearing on the guilt or innocence of the suspect.

Criminal investigation is not science. Investigation does involve a high degree of skill and expertise. The details uncovered by investigation are used as evidence in virtually every trial. Expert testimony by investigators in criminal trials is very common. When found to be relevant and based on reliable methodology, such evidence and testimony are universally accepted as a viable part of the proceedings in court. This still does not mean, however, that investigation is science. Unraveling the case and determining what is significant and relevant will always depend on the skill of the investigator. Each case is different, and there is an infinite variety of information that may be available to be discovered. There are no standardized algorithms or procedures that will solve every case. There will never be a time when we can simply feed all of the facts about a case into a computer, and the computer will tell us what is significant or how the case is to be solved. Although investigation is not science, investigation contributes substantially to legal proceedings, and the evidence and expert testimony provided by investigators will continue to be a valuable part of the process. The present invention involves certain critical aspects of the investigation that precedes the application of Brain Fingerprinting® science, as well as Brain Fingerprinting® science and other scientific and non-scientific procedures and apparatus.

Some research scientists have expressed the view that if we just conduct enough research, and define and study a nearly infinite panoply of parameters, someday the process of investigation will become a scientific process that can be accomplished by applying a set algorithm which does not depend on the skill and judgment of the investigator. There is no reason to believe—or to hope—that this will ever happen. It is our view that science will not, and should not, take the place of skilled criminal investigation. The infinite variety of factors in a crime, and the intimate involvement of human beings in every aspect of the crime, insure that the judgment and skill of the investigator will always be a necessary ingredient in criminal investigations. Science will never make skilled investigators obsolete, and should not attempt to do so.

The process of determining whom to test as a suspect and which items to use as probe stimuli in the Brain Fingerprinting® test will depend on the skill and judgment of the investigator, and will never be accomplished just by applying some set scientific algorithm in the absence of human judgment. Ultimately, the judge and jury will decide whether the evidence uncovered by the investigator and embodied in the probe stimuli is convincing regarding the guilt or innocence of the suspect.

The successful outcome of the investigation is to identify a suspect and probe stimuli that meet specific criteria. The investigation, if successful, will identify one or more suspects, and a number of probe stimuli that meet the necessary criteria to structure a Brain Fingerprinting® test. If the necessary probe stimuli are obtained through investigation, a Brain Fingerprinting® test can be applied, and the suspects can become subjects for the test. Probe stimuli are facts relevant to the crime or situation under investigation that meet the following criteria:

1. Probe stimuli are salient features of the crime that the perpetrator encountered in the course of committing the crime (or other investigated situation).

2. The suspect denies knowing the probe stimuli as features of the crime, based on a claim that he did not participate in the crime.

3. The suspect denies recognizing the probe stimuli as significant or distinguishable from irrelevant information.

4. The suspect claims he does not know the information contained in the probes as a result of exposure after the crime (i.e., either that there was no such exposure, or that any such exposure was minor enough that it did not result in his now knowing the specific information embodied in the probes).

If the investigation is successful in identifying a suspect and probe stimuli that meet the necessary criteria, then the process can proceed to the scientific phase, and a Brain Fingerprinting® test can be conducted.

Phase 2: Scientific Testing with Brain Fingerprinting® Technology

It is in the Brain Fingerprinting® test where science contributes to the process. Brain Fingerprinting® determines scientifically whether or not specific information is stored in a specific person's brain.

Brain Fingerprinting® is a standardized scientific procedure. The input for this scientific procedure is the probe stimuli, which are formulated in the investigation and the interview. The output of this scientific procedure is a determination of “information present” or “information absent” for those specific probe stimuli, along with a statistical confidence for this determination. This determination is made according to a specific, scientific algorithm, and does not depend on the subjective judgment of the scientist.

Brain Fingerprinting® tells us the following: “These specific details about this crime are (or are not) stored in this person's brain.” On the basis of this and all of the other available evidence, a judge and jury make a determination of guilty or innocent.

It has been proven in the scientific arena, and also in court, that the science of Brain Fingerprinting® has the following attributes:

1. This science is testable and has been tested;

2. This science has been peer reviewed and published;

3. This science is accurate, has an error rate extremely close to zero, and has standard procedures for its application;

4. This science is well accepted in the relevant scientific community.

Brain Fingerprinting® determines scientifically what information is stored in a person's brain. It does not determine how that information got there. In order for a determination that certain information is (or is not) stored in a suspect's brain to be useful to a judge and jury, the significance of this finding with regard to the crime must be established. This is accomplished by the investigation and the interview, not by the Brain Fingerprinting® test itself. Brain Fingerprinting® is similar to other sciences in this regard. For example, as discussed above, a fingerprint test can determine that a suspect's fingerprints match the fingerprints found at the scene of the crime, but the fingerprint test does not tell us whether that was because the suspect is guilty or because he was at the scene for a legitimate reason before the crime.

Brain Fingerprinting® scientific testing determines scientifically what information is stored in a person's brain. Brain Fingerprinting® science—or any other science—does not tell us what should, could, or would be stored in a person's brain under what circumstances. Again, it simply tells us what is stored in a person's brain. Providing features of the crime for use as probe stimuli is the domain of investigation—not of science. Deciding whether a person who has or does not have certain specific information stored in his brain committed a crime or not—i.e., whether the true perpetrator would, should, or could have certain information—is a decision to be made by the judge and jury, and is outside the realm of science. Discovering the salient features of the crime that are embodied in the probe stimuli is a function of the investigation, and, although it can be skillfully and effectively done, is outside the realm of science. The determination of whether a person is innocent or guilty based on his knowledge or lack of knowledge of this information is the domain of the judge and jury, and is also outside the realm of science.

Science can only provide scientific data—in the case of Brain Fingerprinting®, a determination of “information present” or “information absent” regarding specific details of a crime in a specific brain. Science, no matter how accurate and valid, is only useful in the context of an effective investigation. For example, the investigator must determine whether or not there is some innocent, legitimate reason why a suspect's fingerprints are at the scene of the crime in order for the scientific finding of a fingerprint match to have value in legal proceedings. In the absence of adequate investigation, science—no matter how accurate and valid—may have little weight in the judicial process. Brain Fingerprinting® is no exception. The investigation and interview that precede the scientific Brain Fingerprinting® test are necessary to provide the information to test, and to provide a background as to the significance of the test vis-à-vis the crime once the scientific results are obtained. The weight of the Brain Fingerprinting® evidence, and its value to the judge and jury in making their determination, depend in part on the effectiveness and skillful execution of the investigation that precedes the scientific testing.

In understanding Brain Fingerprinting® testing or any other scientific procedure, and in distinguishing between scientific procedures and non-scientific procedures that nevertheless involve skill and expertise, it is important to distinguish between two fundamentally different factors: 1) the requirement for skill, intelligence, education, and training on the part of the practitioner; and 2) whether the outcome depends on the subjective judgment of the practitioner.

For example, a nuclear physicist must have considerable skill, intelligence, education, and training to properly conduct a physics experiment. An individual lacking these factors will be unable to conduct a physics experiment successfully. This does not imply, however, that the outcome of a nuclear physics experiment depends on the subjective judgment of the scientist. The result of the experiment depends only on proper and skillful conduct of the science, not on the subjective judgment of the scientist. Two qualified scientists conducting the same experiment will find the same outcome. The same is true of Brain Fingerprinting® testing. A scientist must be skilled, intelligent, educated, and properly trained to successfully conduct a Brain Fingerprinting® test. The outcome of Brain Fingerprinting® test, however, does not depend on the subjective judgment of the scientist, but only on properly conducting the test.

It also takes skill, intelligence, education, and training to be a successful clinical psychologist or counselor. Unlike science, however, the outcome of a clinical counseling session depends on the subjective judgment of the counselor. Two different counselors might end up with two entirely different outcomes when faced with the same situation.

Similarly, criminal investigation requires skill, intelligence, education, and training, and the outcome of a criminal investigation does depend on the skill and judgment of the investigator. Two different investigators may investigate the same crime and focus on different features of the crime, which will be embodied in probe stimuli, or different avenues to seek its solution. As described above, no amount of experience or data collection will transform the process of investigation into a science that is independent of the judgment of the investigator.

Classification Guilty Knowledge Test (CGKT)

In one embodiment of the Classification Guilty Knowledge Test (CGKT), as described in detail below, critical relevant questions, are presented in a series mixed in with known relevant and irrelevant questions. The critical relevant questions a) are relevant to the situation under investigation, and consequently can be expected to be known by the subject if he participated in the situation under investigation, and b) contain information that the suspect claims not to know and would have no known way of knowing unless he had participated in the situation. Critical relevant questions are similar to the relevant questions presented in conventional polygraph guilty knowledge tests and analogous to the probe stimuli presented in Brain Fingerprinting® tests, which are described in U.S. Pat. Nos. 5,363,858, 5,406,956, and 5,467,777, and U.S. patent application Ser. Nos. 10/163,525 and 10/213,089, all by the present inventor.

Some embodiments of the invention deal with a Classification Guilty Knowledge Test (CGKT) for the purposes of “lie detection.” The CGKT detects psychophysiological responses to questions regarding information that a subject may or may not possess regarding a specific situation under investigation.

Embodiments of the invention solve several fundamental problems inherent in currently available polygraph techniques by introducing a more sound scientific basis for drawing conclusions regarding psychophysiological data, as well as a more systematic and objective method for making determinations regarding truth/deception or presence/absence of guilty knowledge. Unlike the conventional CQT, GKT, and R/I tests, the proposed system does not depend on comparing different responses and deciding which one is “larger” or “more of a response.” Rather, it rests on the scientifically much more sound principle of classification. One response, the response to the critical relevant questions, is classified as being in one of two different categories.

To accomplish this, a new type of question is presented, the known relevant question described in detail below, in addition to the two types of questions that have been used in conventional polygraphy, namely relevant (here, “critical relevant”) and irrelevant. The subject is presented with two types of questions, the responses to which will form the standards for the classification: 1) irrelevant questions and 2) known relevant questions.

Consider, for example, an investigation in which the examiners know that John Jones was killed with a knife by the river. The suspect knows that he is being investigated for the murder of John Jones, and knows that Jones was killed down by the river, but claims not to know what the murder weapon was.

A known relevant question could be, “Do you know that Jones was killed by the river?” A critical relevant question could be, “Do you know that Jones was killed with a knife?” An irrelevant question could be, “Do you know that Jones was killed with a shotgun?” The alternatives can also be presented in the following way:

“Regarding the murder weapon, do you know that it was a:”
“knife” (critical relevant)
“shotgun” (irrelevant)
“axe” (irrelevant)

“Regarding the location of the murder, was it:”
“by the river” (known relevant)
“at the gas station” (irrelevant)
“by the freeway” (irrelevant)

The responses to the critical relevant questions are classified as being either more similar to the known relevant responses or more similar to the irrelevant responses. If the critical relevant responses are more similar to the known relevant responses, then this provides evidence that the subject does indeed know the critical relevant information (e.g., knows details about the crime that he would have no way of knowing unless he were indeed a participant in the crime). This classification can be accomplished in a highly objective and scientific way through known mathematical classification techniques such as bootstrapping (see U.S. Pat. Nos. 5,363,858, 5,406,956). Moreover, a statistical confidence for the result obtained in each specific case can be readily computed, as described in the referenced patents. This allows for a more objective and scientific determination than has been available in the previous art.

Classification, as opposed to mere comparison, can be appropriately applied because the system embodies the scientific prerequisites for a classification task: 1) two standards for data of two specific, distinct categories; 2) an experimental design that produces data that fit either into one category or the other. The psychophysiological responses to the irrelevant questions provide a standard for the subject's responses to unknown information: details that are not known to the subject to be correct details about the investigated situation, although they are plausible if the subject does not know the correct details. The psychophysiological responses to the known relevant questions provide a standard for the subject's responses to known, correct details about the investigated situation. The psychophysiological responses to the critical relevant questions provide data that are of one category if the subject knows the critical relevant details of the investigated situation, and are of the other category if the subject does not know the critical relevant details of the investigated situation. If the subject knows the details about the investigated situation that are contained in the critical relevant questions, then the critical relevant questions are like the known relevant questions: both contain known details about the investigated situation. If the subject does not know the details about the investigated situation contained in the critical relevant questions, then the critical relevant questions are like the irrelevant questions: they contain details the subject does not know or recognize as being correct details about the situation, although they, like the irrelevant questions, contain information that is plausible.

Thus, the system employs three types of questions: critical relevant questions, known relevant questions, and irrelevant questions. The subject may reply with a “yes” or “no” response to each question by, e.g. pressing one of two designated buttons on the device 160. Questions are asked in a standardized manner, and operator bias in stimulus presentation may be eliminated by using the speech synthesizer 131 to aurally present questions to the subject based on previously prepared text-based questions. Button-press responses are monitored and recorded by the system 100.

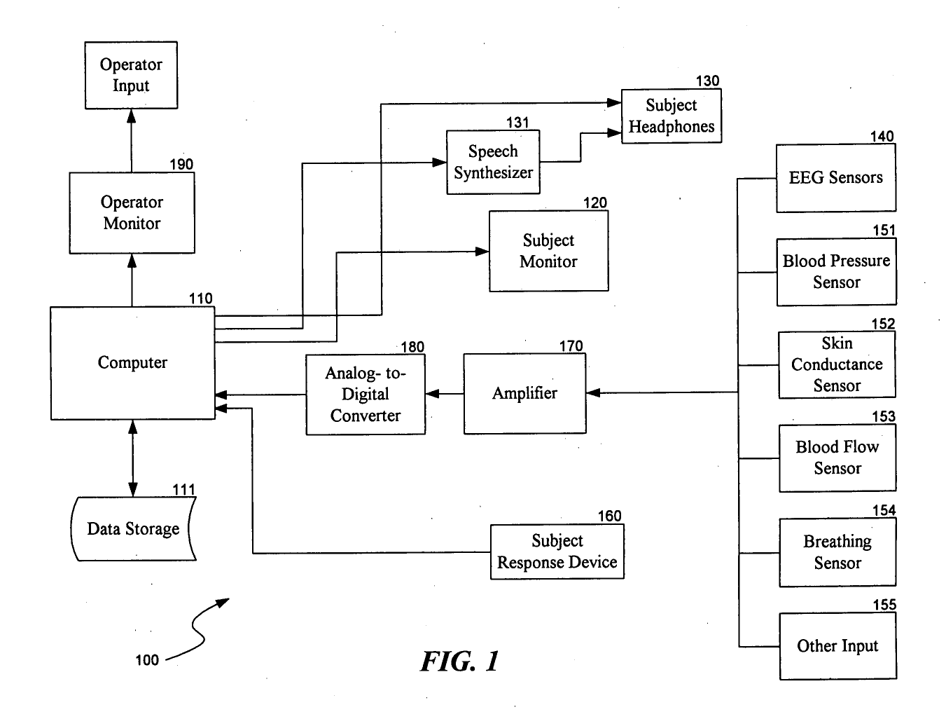

FIG. 1 illustrates the components of a Classification Guilty Knowledge Test (CGKT) system 100. Although not required, aspects and embodiments of the invention are described in the general context of computer-executable instructions, such as routines executed by a general-purpose computer, e.g., a server or personal computer. Those skilled in the relevant art will appreciate that aspects of the invention can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The invention can be embodied in a special purpose computer or data processor that is specifically programmed, configured or constructed to perform one or more of the computer-executable instructions explained in detail below. Indeed, the term “computer”, as used generally herein, refers to any of the above devices, as well as any data processor.

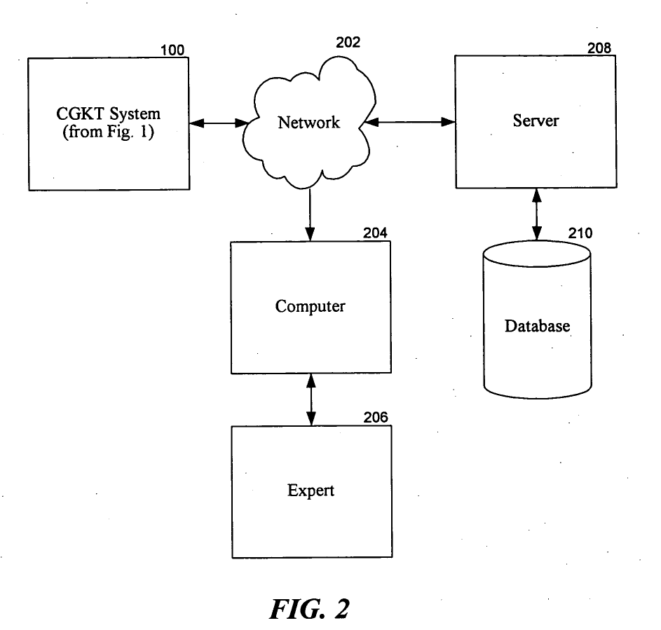

As explained below with respect to FIG. 2, the invention can also be practiced in distributed computing environments, where tasks or modules are performed by remote processing devices, which are linked through a communications network, such as a Local Area Network (“LAN”), Wide Area Network (“WAN”) or the Internet. In a distributed computing environment, program modules or sub-routines may be located in both local and remote memory storage devices. Aspects of the invention described below may be stored or distributed on computer-readable media, including magnetic and optically readable and removable computer discs, stored as firmware in chips (e.g., EEPROM chips), as well as distributed electronically over the Internet or over other networks (including wireless networks). Those skilled in the relevant art will recognize that portions of the invention may reside on a server computer, while corresponding portions reside on a client computer. Data structures and transmission of data particular to aspects of the invention are also encompassed within the scope of the invention.

A computer 110 generates visual stimuli that are displayed to the subject by a subject monitor 120. The computer 110 generates non-speech sounds that are presented to the subject through a set of subject headphones 130. The computer generates the language stimuli that are converted to speech by an optional speech synthesizer 131 and presented to the subject through the subject headphones 130. Alternatively, the computer 110 can directly generate the speech sounds and present them through the subject headphones 130.

An optional set of EEG sensors 140 collects EEG data originating in the subject's central nervous system (as described below). Four other sensors collect psychophysiological data arising from the functioning of the subject's autonomic nervous system. A blood pressure sensor 151 collects blood pressure data. A skin conductance sensor 152 collects skin conductance data. A blood flow sensor 153 collects data on the subject's blood flow. A breathing sensor 154 collects data on the subject's breathing. A subject response device 160 collects data on the subject's overt responses (e.g., button presses in response to questions or stimuli, or a microphone to receive verbal responses). An amplifier 170 amplifies the signals from the sensors. An analog-to-digital (ADC) converter 180 converts these signals to digital data. The computer 110 displays the data and the stimuli to the operator on an operator monitor 190. The computer analyzes the data and displays the results to the operator on the operator monitor 190. The computer reads data from and stores data to a data storage device 111. For example, the data storage device 111 stores data for questions and data corresponding to subject responses from all input devices 140, 151-155 and 160.

The computer 110 may also include additional sensors or subject input devices 155 to receive subject data for any of the following: a) skin resistance, b) cardiovascular measurements, c) plethysmographic measurements, d) electrocardiogram (EKG), e) cardiac systolic time intervals, f) measurements involving the output of the vocal cords during speech, g) voice stress analysis, h) eye measurements, i) eye movement measurements, j) pupil diameter k) measurement of brain activity, l) measurement of cerebral blood flow, m) measurement of the level of activation of at least one specific part of the brain, n) at least one of magnetic resonance imaging (MRI) and functional MRI, o) brain imaging, p) tomography, or q) tomography using at least one of visible light and laser light. Many types of sensors or instruments are available to obtain such data, as those skilled in the relevant art will appreciate.

The system 100 obtains the standard measurements used in conventional polygraphy: skin conductance, breathing activity, and continuously monitored blood pressure, as well as peripheral blood flow as measured by a photoplethysmograph from the sensors 151-155. The measurements are stored in the data storage device 111. These recorded psychophysiological responses to the questions may then be classified using bootstrapping statistics. The critical relevant responses are classified as being more similar either to the known relevant responses or to the irrelevant responses. A statistical confidence for this determination may be computed using bootstrapping statistics or the like.

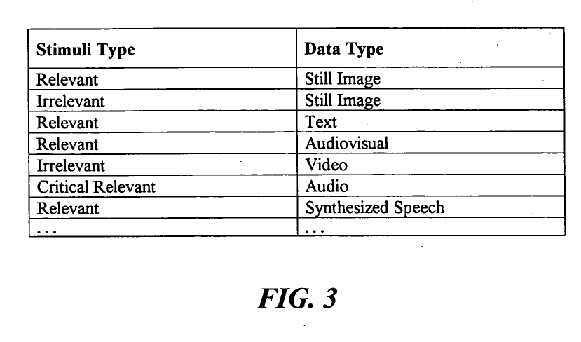

Referring to FIG. 3, an example of a data structure or record for use by the system 100 is shown. The record includes multiple stimuli, such as questions, for each of the three types: relevant, irrelevant, and critical relevant stimuli. Associated with each of the stimuli is a piece of data that can take any of multiple forms, such as still images, text, video, audio, audiovisual, etc. Some of the data may be converted from other data, such as synthesized speech converted from text. Each of the stimuli may be associated with an appropriate field associating the piece of data with the stimuli type, as shown in FIG. 3. While the term “field” and “record” are used herein, any type of data structure can be employed. For example, relevant data can have preceding headers, or other overhead data preceding (or following) the relevant data. Alternatively, relevant data can avoid the use of any overhead data, such as headers, and simply be recognized by a certain byte or series of bytes within a serial data stream. Any number of data structures and types can be employed herein.

While only a few stimuli are shown in FIG. 3, any given investigation may include numerous questions or pieces of data. A separate data structure containing all stimuli for a given inquiry may be assembled as a combined data structure. Additional data may also be provided in the data structure, such as responses from a subject associated with each stimulus, classification based on such responses, statistical data, and so forth, as described. Alternatively, a separate data structure or record may be created and stored for all responses from a subject.

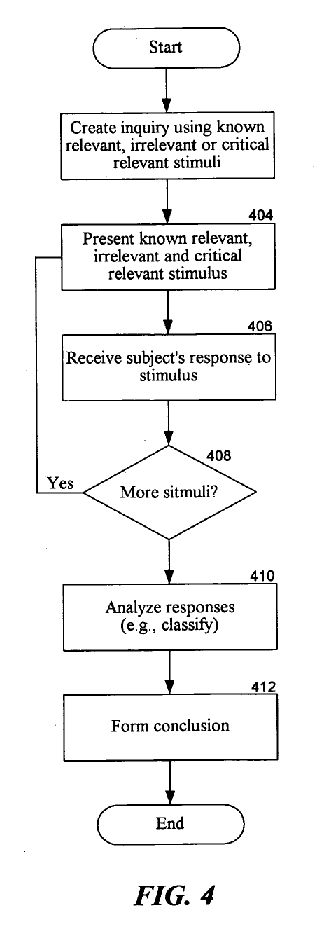

Referring to FIG. 4, an example of a routine for conducting an investigation is shown. Beginning in block 402, a operator or investigator creates content for an inquiry by assembling or creating known relevant, irrelevant and critical relevant stimuli. The stimuli may then be arranged in a predetermined order, random order, and so forth.

In block 404, one of the stimuli is presented to the subject, and in block 406, the system 100 receives and stores the subject's response to the stimulus. In block 408, the routine determines whether more stimuli exists, and if so, loops back to again performing blocks 404, 406 and 408. If no additional stimuli exist (e.g., no additional fields in the data structure exist), then the system 100 analyzes the stored responses. The system may classify the responses, as described. Finally, in block 412, a conclusion is reached regarding the analyzed responses.

Of course, additional functions may be employed. For example, each response from a subject can be analyzed to determine if it is acceptable, or if it should be discarded. For example, if the subject moved his head during testing, this could produce a response contaminated by artifacts that should be discarded or later corrected. Therefore, an additional stimulus or question may be introduced so that a total number of desired, artifact-free responses is obtained.

If the critical relevant responses are more similar to the known relevant responses, then the conclusion that follows is that the information contained in the critical relevant stimuli, like the information contained in the known relevant stimuli, is crime-relevant information that is known to the subject. The determination is “deceptive” (assuming that the subject claims not to know the critical relevant information and answers “no” to the critical relevant questions), and the conclusion is that the subject knows the “guilty knowledge.” If the critical relevant responses are more similar to the irrelevant responses, then the determination is that the subject is “nondeceptive” in denying the guilty knowledge. The information contained in the critical relevant questions, like the information contained in the relevant questions, is not known by the subject to be relevant to the crime. (This again is assuming that he claims not to know the critical relevant information and answers “no” to the critical relevant questions). Thus, under one system, only psychophysiological (or autonomic nervous system) responses are analyzed, and the system 100 may omit the EEG data.

In an alternative embodiment, central nervous system (CNS) activity is monitored along with autonomic nervous system (ANS) activity. That is, the system 100 measures EEG or other CNS activity in addition to ANS measures such as skin conductance, breathing, and blood pressure. Cognitive as well as emotional responses are measured and included in the analysis. This may serve to combine the strengths of “Brain Fingerprinting®” technology noted above, which measures CNS activity, and conventional polygraphy, which measures ANS activity. (In general, alternatives and alternative embodiments described herein are substantially similar to previously described embodiments, and common elements and functions are identified by the same reference numbers. Only significant differences in construction or operation are described in detail.)

Some embodiments of the invention involve Brain Fingerprinting® technology. Brain Fingerprinting® technology is a scientific technology that directly detects the presence or absence of information in the brain as that information is processed by the central nervous system (CNS). It does not deal directly with detection of deception or lie detection. No questions are asked, and no answers are given, during a Brain Fingerprinting® test. The results of a Brain Fingerprinting® test, “information present” or “information absent”, may be identical whether or not the person lies at any time about the specific information in question or any other subject.

No questions need to be asked or answered during Brain Fingerprinting® testing. Structurally, Brain Fingerprinting® tests involve measuring electrical brain activity while the subject views specific stimuli. The test reveals not the truth or falsehood of any testimony, but simply the presence or absence of information stored in the brain. Unlike polygraphy, Brain Fingerprinting® technology is non-testimonial. In this regard Brain Fingerprinting® technology is similar to DNA testing and fingerprinting. Moreover, Brain Fingerprinting® technology is non-invasive.

An alternative to asking critical relevant questions is to present words, phrases, pictures, information, or items that are relevant to the investigated situation in a non-question format. Similarly, known relevant and irrelevant information may be presented in the form of questions, words, phrases, pictures, information, or items. The responses to the critical relevant questions are classified as being either more similar to the known relevant responses or more similar to the irrelevant responses. If the critical relevant responses are more similar to the known relevant responses, then this provides evidence that the subject does indeed know the critical relevant information (e.g., knows details about the crime that he would have no way of knowing unless he were indeed a participant in the crime).

Combining CNS measurements, such as those typically used in Brain Fingerprinting® with ANS measures, such as those used in standard polygraphy may provide more useful results than either CNS or ANS measurements alone, particularly when ANS and CNS data are combined any suitable analysis algorithm to provide a single determination. CNS and ANS data may be combined in any of several ways. One is stepwise linear discriminant analysis, which has been used with brain waves before. Several measures, such as skin conductance and cardiovascular measures, could be used as additional predictors, in addition to the brain data. Those skilled in the art would recognize several other ways to accomplish a similar result, such as converting the several measures to z-scores and use bootstrapping statistics on the sum of the z-scores. Still another method would be to rank order individual trial scores on each measure and compute bootstrapping statistics on the sum of the ranks for the several measures.

Whereas Brain Fingerprinting® technology detects information stored in the brain, polygraphy, including some embodiments of the CGKT, attempts to detect deception or lying. In polygraphy, one seeks to gain information by interrogating the subject, noting the answers given, and attempting to discern whether or not these answers are truthful. In some embodiments of the CGKT (and the conventional GKT), the questions asked are about “guilty knowledge” that the subject may or may not have. If the subject claims not to have the guilty knowledge, and the physiological responses are interpreted as to indicate that the subject is lying when making this claim, then the conclusion is that the person does have the guilty knowledge.

Like all polygraph tests, some embodiments of the CGKT measures physiological arousal mediated by the autonomic nervous system. The ability to measure this physiological arousal accurately is a prerequisite for a polygraph test to work. Accurate measurement of physiological arousal, however, is not enough. The goal of polygraphy is to produce emotional and corresponding physiological arousal differentially, depending on whether or not the subject is lying, and then to measure and correctly interpret this difference. Since Brain Fingerprinting® technology simply measures the presence or absence of information stored in the brain as it is processed by the central nervous system, it is not necessary to structure a test to elicit any particular emotions. Differences in emotional response patterns, whether they are of cultural, religious, psychological, or any other origin, do not affect the test. Since the CNS brain responses measured by Brain Fingerprinting® technology take place at the moment of recognition of the stimulus and before the subject even starts thinking about structuring or faking a response, Brain Fingerprinting® technology is extremely resistant to many imaginable kinds of countermeasures.

Since Brain Fingerprinting® technology is strictly a method to develop scientific data and does not involve interrogation, Brain Fingerprinting® technology experts typically have a different skill set than that typically possessed by polygraphers. Brain Fingerprinting® experts are scientists, and not necessarily interrogators. Polygraphers, who use the polygraph as an adjunct to interrogation, are typically skilled interrogators. The CGKT provides a methodology whereby polygraphers with their existing skill sets and existing equipment can improve their ability to determine if a suspect is lying regarding whether or not he has knowledge about a crime or investigated situation. Although this information lacks the high scientific validity, statistical confidence, objectivity, and admissibility in court enjoyed by Brain Fingerprinting® testing results, it can nevertheless be of use in structuring a successful interrogation and eliciting a confession from a guilty party, and in guiding examiners to apply their resources towards those more likely to be guilty and away from those more likely to be innocent. The CGKT has the advantage of being able to be administered with more readily available equipment and by more readily available personnel, at least until such time as Brain Fingerprinting® technology equipment and trained Brain Fingerprinting® technology experts become widely available. In sum, use of CNS data from Brain Fingerprinting® with ANS data in this alternative embodiment provides numerous advances over either one individually.

In another alternative embodiment, one or more subjects are presented with an ongoing audiovisual presentation that is largely irrelevant to the situation under investigation. Details about the investigated situation are embedded in the presentation. Some of these details are known by examiners to be known to the subject (e.g., details of the crime that are disclosed in news media reports that the subject has seen). These constitute the known relevant stimuli. Some of these embedded details are critical relevant stimuli, details that the subject has encountered if he participated in the specific crime or other event being investigated, but has no other way of knowing. The irrelevant stimuli are simply events in the audiovisual presentation that have nothing to do with the investigated situation. The system 100 continuously monitors the subject's psychophysiological responses, and specific responses to these three types of stimuli are extracted from the continuous data stream by computer analysis. A computerized data-analysis algorithm then compares the responses to the respective stimulus types, and a determination is made as to the presence or absence of the incriminating information in the subject's brain. The algorithm must distinguish between the brain responses to the different stimulus types, and in particular to determine if the responses to the critical relevant stimuli are more similar to the responses to the known relevant stimuli or to the responses to the irrelevant stimuli. These comparisons can be accomplished by several techniques, including correlation, coherence analysis, spectral analysis, dynamical systems analysis, or any of these in combination with bootstrapping.

In a modification of this alternative embodiment, the system 100 omits the irrelevant stimuli, and analyzes only the known relevant and critical relevant stimuli, or even only the critical relevant stimuli. The three-stimulus paradigm described herein, however, provides more statistical power, more experimental control, higher validity, and higher accuracy than an algorithm in which only one or two stimulus types (or question types) are included in the analysis.

In another embodiment, embedded responses could be measured remotely or covertly, without the subject even knowing that he was being monitored. Hamlet the, “The play's the thing, wherein I'll catch the conscience of the king.” (Hamlet II, ii, 617.) To gather data on his uncle's possible guilt in the murder of his father, Hamlet presented a play in which a murder was committed with similar details to the murder of his father, and watched his uncle's response to the play. In this embodiment of the invention, a similar stimulus (the critical relevants), is presented, and is similarly embedded. In addition, two other types of stimuli are embedded for the sake of a precise classification of responses. Also, unlike Hamlet's ploy, this invention can take into account not merely responses that are apparent through simple observation, but covert psychophysiological responses. Moreover, the invention uses a computer analysis to arrive at an objective and accurate determination. In this embodiment, an audiovisual presentation can be presented ostensibly for the purpose of entertainment or instruction, and information relevant to a specific crime, terrorist training, or other situation under investigation embedded in it. Responses such as heart rate, skin temperature, and possibly even CNS measures can be monitored remotely, with or without the subject's knowledge, with appropriately sensitive sensors. Computer analysis of the responses can filter noise, amplify the signal to noise ratio, and distinguish accurately between a person who has information indicative of participation in the event in question and one who lacks that knowledge.

Under such an alternative embodiment, the system 100 can employ sensors hidden in an article of clothing, such as a hat or eyeglasses, or in other articles that are worn on the head such as a earphones or jewelry. The subject would thus wear such a hat and provide responses to the system while stimuli are presented to him. Indeed, an audience of subjects can wear such items, and the system 100 can obtain responses from all subjects either with, or without, their knowledge.

Alternatively or additionally, the system 100 continuously monitors ANS and/or CNS activity during the course of an interrogation or negotiation. The information gathered is used to inform the interrogator or negotiator of the covert emotional and/or cognitive state and activities of the subject. This information is used to guide the course of the interrogation or negotiation. This information need not have anything to do with whether or not the subject is lying. In this embodiment, the system is used not for detection of deception, but for detection of the covert emotional and cognitive processes of the subject. For example, in an interrogation or negotiation, it may be useful for the interrogator to know that whenever a subject's relationship with a particular person is discussed, this causes emotional and corresponding physiological arousal. From this, the interrogator or negotiator can infer that this specific relationship is an emotionally charged issue for the subject. This information may be useful in guiding the interrogation or negotiation towards or away from this subject, depending on the goals of the interrogation or negotiation. It may also be useful information when offering consideration to the subject in return for consideration cooperation of one kind or another to know what the subject's “hot buttons” are. Similarly, it may be useful to the interrogator or negotiator to know which subjects of discussion cause the subject to engage in more intense cognitive, CNS activity. If the interrogator or negotiator can discern psychophysiologically that a person has to think hard about his answer to a particular question or his response to a particular assertion, offer, or statement, this information can be useful in achieving a successful outcome. The psychophysiological measurements can be taken remotely, either covertly or with the subject's knowledge.

The simplest time measurement is the time between the stimulus and the subject's response. More sophisticated time measurements, however, may provide more useful data. Such more sophisticated measurements include, for example, the time course of positive and negative voltage changes in event-related brain potentials, phasic changes in the frequency domain of EEG signals, and changes in the timing of cardiac signals.

Referring to FIG. 2, an alternative embodiment 200 is shown. A network 202 (e.g. the Internet or World Wide Web (“Web”)) receives data from (such as data from a test subject), and provides data to (such as test data) the system 100. While the Internet is shown, a private network, such as an intranet may likewise be used herein. The network may have a client-server architecture, in which a computer is dedicated to serving other client computers, or it may have other architectures such as a peer-to-peer, in which one or more computers serve simultaneously as servers and clients. A remote computer 204, coupled to the network 202, may receive the test data, and thus permit a remotely located doctor or other individual to analyze and interpret the test data.

At least one server computer 208, coupled to the network 202, may likewise receive the test data, and store such data in a database or databases 210. The server computer 208 may analyze the data and provide enhancements to the CGKT, testing, or other enhancements. Likewise, the server computer can provide software updates to the system 100, such as updates based on such data analysis. Data analysis of large data sets may help to evaluate and improve the data analysis, stimulus presentation, and data acquisition algorithms embodied in the system, and to refine the procedures for applying the invention. While not shown, the server computer(s), including the database(s), may employ security measures to inhibit malicious attacks on the system, and to preserve integrity of the messages and data stored therein (e.g., firewall systems, secure socket layers (SSL) password protection schemes, encryption, and the like).

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list, and any combination of the items in the list. Where the context permits, words in the above Detailed Description using the singular or plural number may also include the plural or singular number respectively.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. The teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above detailed description. While the above description details certain embodiments of the invention and describes the best mode contemplated, no matter how detailed the above appears in text, the invention can be practiced in many ways. Details of configurations, functions, etc. may vary considerably in implementation details, while still being encompassed by the invention disclosed herein. As noted above, particular terminology used when describing certain features, or aspects of the invention should not be taken to imply that the terminology is being re-defined herein to be restricted to any specific characteristics, features, or aspects of the invention with which that terminology is associated. In general, the terms used should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

While certain aspects of the invention are presented below in certain claim forms, the inventors contemplate the various aspects of the invention in any number of claim forms. For example, while only one aspect of the invention is recited as embodied in a computer-readable medium, other aspects may likewise be embodied in a computer-readable medium. Accordingly, the inventors reserve the right to add additional claims after filing the application to pursue such additional claim forms for other aspects of the invention.

1. An apparatus for detecting deception by a test subject, information possessed by the test subject, or both, the apparatus comprising:
at least one data output device to provide testing data to the test subject;
at least one data input device to receive responses from the test subject based on the testing data; and
at least one computer, associated with at least one data storage device, and coupled among the at least one data output device and the at least one data input device;
wherein the testing data includes:
known relevant stimuli that are relevant to a specific subject matter related to specific knowledge, events, or actions, and are known to the test subject;
irrelevant stimuli that are irrelevant to the specific subject matter and
critical relevant stimuli that are relevant to the specific subject matter and that the test subject does not acknowledge knowing;
wherein the computer receives from the at least one input device, and stores in the data storage device, measurements of the test subject's psychophysiological responses to the testing data,
wherein the stored measurements permit classifying the test subject's responses to the critical relevant stimuli as being more similar to the test subject's responses to the known relevant stimuli or more similar to the test subject's responses to the irrelevant stimuli to detect deception by a test subject, information possessed by the test subject, or both.

2. The apparatus of claim 1 wherein the at least one input device includes:
a subject-actuated input device for receiving subject-selected responses to stimuli in the testing data;
at least one electroencephalogram (EEG) sensor; and
at least two different autonomic-nervous system sensors selected from the following group: blood pressure sensor, skin conductance sensor, blood flow sensor, and breathing sensor;
wherein the computer receives data from the EEG sensor to determine recognition or processing by the test subject's brain of at least some of the testing data;
wherein the testing data includes at least visual or auditory test data, and wherein the at least one output device includes at least one of:
a display device for providing the visual test data to the test subject;
an auditory output device for providing the auditory test data to the test subject; and
a speech synthesizer coupled between the computer and the auditory output device to provide synthesized speech to the test subject via the auditory output device.

3. The apparatus of claim 1, further comprising:
a operator station, wherein the operator station includes at least a display device coupled to the computer, and an operator input device for receiving commands from an operator.

4. The apparatus of claim 1, further comprising:
a remote computer, coupled to the at least one computer via a network, wherein the remote computer includes at least a display device coupled to the remote computer, and wherein the remote computer receives at least the measurements of the test subject's psychophysiological responses to the testing data to classify the test subject's responses to the critical relevant stimuli as being more similar to the test subject's responses to the known relevant stimuli or more similar to the test subject's responses to the irrelevant stimuli.

5. A method for detecting deception by a subject, information possessed by the subject, or both, the method comprising:
presenting a subject with testing data, wherein the testing data includes:
known relevant stimuli that are relevant to specific subject matter, wherein the known relevant stimuli are associated with the specific subject matter and are known to the subject;
irrelevant stimuli that are irrelevant to the specific subject matter; and
critical relevant stimuli that are relevant to the specific subject matter and that the subject does not acknowledge knowing;
receiving data representing the subject's psychophysiological responses to the testing data, and,
analyzing the subject's responses to the critical relevant stimuli as being more similar to the subject's responses to the known relevant stimuli or more similar to the subject's responses to the irrelevant stimuli, wherein the analyzing at least assists in determining whether the subject's responses to the testing data indicate deception or a presence of information known to the subject.

6. The method of claim 5 wherein receiving data includes receiving from the subject overt responses to the testing data through verbal responses or non-verbal, motor responses.

7. The method of claim 5 wherein the testing data includes questions, and wherein the subject is instructed to lie in response to the known relevant stimuli and tell the truth in response to the irrelevant stimuli.

8. The method of claim 5 wherein the testing data includes questions.

9. The method of claim 5 wherein the testing data includes audiovisual data or simultaneously presented auditory and visual stimuli.

10. The method of claim 5 wherein the subject is not given instructions to respond overtly and voluntarily, but simply perceives at least a portion of the test data and involuntarily produces psychophysiological responses thereto, due content of the test data, wherein the psychophysiological responses represent knowledge that the subject has or knowledge that the subject lacks regarding matters to which at least some of the test data are relevant.

11. The method of claim 5 wherein presenting the subject with testing data includes providing the known relevant, irrelevant and critical relevant stimuli to the subject as embedded in a series of still images or video, and wherein a substantial portion of the still images or video comprises information unrelated to the specific subject matter.

12. The method of claim 5 wherein presenting the subject with testing data includes providing the known relevant, irrelevant and critical relevant stimuli to the subject as embedded in a series of visual, auditory or audiovisual test data.

13. The method of claim 5 wherein presenting the subject with testing data includes providing the subject with text data, and still images or video.

14. The method of claim 5 wherein receiving data includes receiving from the subject psychophysiological responses that include at least one of
a) skin conductance,
b) skin resistance,
c) measurements of breathing,
d) measurements of blood flow,
e) plethysmographic measurements,
f) cardiovascular measurements,
g) blood pressure,
h) electrocardiogram (EKG),
i) cardiac systolic time intervals,
j) measurements involving output of vocal cords during speech,
k) voice stress analysis,
l) eye measurements,
m) eye movement measurements,
n) pupil diameter
o) measurements of brain activity,
p) EEG,
q) measurements of cerebral blood flow,
r) measurements of a level of activation of at least one specific part of the subject's brain,
s) at least one of magnetic resonance imaging (MRI) and functional MRI,
t) brain imaging,
u) tomography, and
v) tomography using at least one of visible light and laser light.

15. The method of claim 5 wherein receiving data includes receiving autonomic nervous system responses from the subject.

16. The method of claim 5 wherein receiving data includes receiving measurements indicative of at least one of a) stress, b) emotion, c) cognitive activity, and d) mental activity, from the subject.

17. The method of claim 5 wherein receiving data includes measuring autonomic-nervous-system-based (ANS) responses and central-nervous-system-based (CNS) responses, and
wherein the method further comprises combining the ANS and CNS responses to make a single determination regarding at least one of a) deception, b) guilty knowledge, and c) information possessed by the subject.

18. The method of claim 5 wherein the subject's psychophysiological responses include autonomic-nervous-system-based (ANS) responses and central-nervous-system-based (CNS) responses.

19. The method of claim 5 wherein analyzing the subject's responses includes classifying the subject's responses with a calculated statistical confidence.

20. The method of claim 5 wherein analyzing the subject's responses includes employing a bootstrapping analysis routine to at least some of the subject's responses.

21. The method of claim 5 wherein analyzing the subject's responses includes combining two or more of the subject's psychophysiological responses.

22. The method of claim 5 wherein presenting the subject with testing data includes:
identifying for the subject at least one category to which at least some of features of the specific subject matter belong;
presenting at least two alternatives related to the identified category, wherein:
the known relevant stimuli represent correct alternatives that match the specific subject matter and are known to the subject regardless of whether the subject participated in subject matter associated with the specific subject matter,
the irrelevant stimuli are incorrect alternatives that do not match the specific subject matter, but are plausible for a subject lacking knowledge of the subject matter, and
the critical relevant stimuli are correct alternatives that match the investigated situation and that the subject does not acknowledge knowing.

23. An apparatus for detecting at least one of a) deception, b) guilty knowledge, and c) information possessed by a subject, the apparatus comprising:
means for presenting the subject with stimuli to be appreciated by at least two senses of the subject;
means for measuring autonomic-nervous-system-based (ANS) psychophysiological responses by the subject in response to at least some of the presented stimuli;
means for measuring central-nervous-system-based (CNS) psychophysiological responses by the subject in response to at least some of the presented stimuli; and
means for analyzing the CNS and ANS responses by combining at least portions of the ANS and CNS responses to facilitate a single determination regarding at least one of a) deception, b) guilty knowledge, and c) information possessed, by the subject.

24. The apparatus of claim 23, further comprising means for receiving subject-directed verbal or motor response from the subject in response to the stimuli.

25. The apparatus of claim 23 wherein the means for analyzing includes calculating a statistical confidence level from the combined ANS and CNS responses.

26. The apparatus of claim 23 wherein the means for means for measuring ANS or CNS responses include at least one sensor, and wherein the apparatus further comprises means for disguising the sensor from the subject.

27. The apparatus of claim 23, further comprising remote computing means for remotely receiving the CNS and ANS responses and providing them to the means for analyzing.

28. A computer-readable medium whose contents embody a data structure for use in detecting deception by a subject, information possessed by the subject, or both, the data structure comprising:
testing data for presentation to the subject, wherein the testing data includes:
at least one known relevant stimulus data structure that represents information relevant to specific subject matter, and wherein the relevant information is known to the subject;
at least one irrelevant stimulus data structure that represents information irrelevant to the specific subject matter; and
at least one critical relevant stimulus data structure that represents information critically relevant to the specific subject matter, and wherein the subject does not acknowledge knowing the critically relevant information.

29. The computer-readable medium of claim 28 wherein at least one of the known relevant stimuli, irrelevant stimuli or critical relevant stimuli data structures include audiovisual information.

30. The computer-readable medium of claim 28 wherein the computer-readable medium is a logical node in a computer network receiving the contents.

31. The computer-readable medium of claim 28 wherein the computer-readable medium is a computer-readable disk.

32. The computer-readable medium of claim 28 wherein the computer-readable medium is a data transmission medium carrying a generated data signal containing the contents.

33. The computer-readable medium of claim 28 wherein the computer-readable medium is a memory of a computer system.

34. A system for detecting deception by a subject, information possessed by the subject, or both, the system comprising:
at least one input device;
at least one output device;
at least one computer coupled among the input and output devices,
wherein the computer is programmed to
present the subject with stimuli in at least one of a) the auditory modality and b) the visual modality, via the at least one output device, wherein the stimuli are presented under a predetermined time course;
measure, via the at least one input device, psychophysiological responses to the stimuli by the subject, and timing of the responses based on the predetermined time course; and
process the responses and provide output for use in detecting at least one of a) deception and b) information possessed by the subject, wherein the processing includes the consideration of the timing or time course of the responses.

35. The system of claim 34 wherein the stimuli include computer-generated speech or digitized speech.

36. The system of claim 34 wherein the stimuli include continuous audiovisual data lasting longer than any individual stimulus of interest and longer than any individual response from the subject.

37. The system of claim 34 wherein the stimuli include at least two of a) irrelevant stimuli, b) known relevant stimuli, and 3) critical relevant stimuli.

38. The system of claim 34, further comprising a data transmitter for transmitting to a remote computer data at least related to the responses.

39. The system of claim 34 wherein the responses includes both overt responses and psychophysiological responses from the subject.

40. The system of claim 34 wherein the responses are used as an aid in conducting at least one of interrogation and negotiation.

41. The system of claim 34 wherein the responses are measured either remotely or covertly.

42. A method for detection of information possessed by a subject regarding specific subject matter, the method comprising:
presenting the subject with at least three types of stimuli, including:
target stimuli that are relevant to the specific subject matter, that were encountered by any individuals who participated in events or actions related to the specific subject matter, and that are known to the subject by i) the subject being exposed subsequent to the events or actions to information connecting the target stimuli to the events or actions, or ii) the subject being informed of the target stimuli and their connection to the events or actions before presenting the subject with the at least three types of stimuli;
probe stimuli that are relevant to the events or actions, that were encountered by any individuals who participated in the events or actions, and that the subject does not acknowledge knowing, denies are significant to him, or denies being able to distinguish from irrelevant stimuli with respect to the specific subject matter;
irrelevant stimuli that are irrelevant to the specific subject matter, wherein some of the irrelevant stimuli are similar to the target stimuli and would be indistinguishable from target stimuli by a person lacking knowledge of the specific subject matter, and wherein some of the irrelevant stimuli are similar to and would be indistinguishable from probe stimuli for a person lacking knowledge of the specific subject matter; and
obtaining psychophysiological responses to at least some of the stimuli.

43. The method of claim 42, further comprising classifying the responses to the probe stimuli as being more similar to the responses to the target stimuli or more similar to the responses to the irrelevant stimuli.

44. The method of claim 42, further comprising drawing at least one conclusion regarding presence or absence of information possessed by the subject.

45. The method in claim 42 wherein the subject is instructed to not make any voluntary or overt responses.

46. The method in claim 42 wherein the psychophysiological responses include measurements of brain activity.

47. The method in claim 42 wherein the psychophysiological responses include EEG measurements.

48. The method in claim 42 wherein the psychophysiological responses include event-related brain potential (ERP) measurements.

49. The method in claim 42 wherein the psychophysiological responses include memory and encoding related electroencephalographic response (MERMER) measurements.

50. A method of at least one of a) interrogation b) negotiation, and c) verbal interaction comprising:
substantially continuously monitoring at least two of the following different psychophysiological measurements in a human subject during a verbal interaction, without regard to a specific, yes-or-no answer to a question:
a) skin conductance, b) skin resistance, c) measurements of breathing, d) measurements of blood flow, e) plethysmographic measurements, f) cardiovascular measurements, g) blood pressure, h) electrocardiogram (EKG), i) cardiac systolic time intervals, j) measurements involving output of vocal cords during speech, k) voice stress analysis, l) eye measurements, m) eye movement measurements, n) pupil diameter, o) ANS measures, p) CNS measures;
substantially continuously or periodically analyzing the at least two different psychophysiological measurements, wherein the analyzing includes combining the at least two different psychophysiological measurements; and
determining at least one of a) emotional responses, b) emotional state, c) cognitive responses, and d) mental state of the human subject based on the analysis to assist in conducting the at least one of a) interrogation b) negotiation, and c) verbal interaction.

51. The method in claim 50 wherein the monitoring is conducted remotely, covertly, or both.
